(19) United States
(12) Patent Application Publication
(10) Pub. No.: US 2008/0097442 A1
(43) Pub. Date: Apr. 24, 2008

(54) METHOD AND DEVICE FOR IMPROVING THE FUNCTION OF TAPER LOCKS USED FOR SPINAL STABILIZATION

(76) Inventor: Robert A. DIXON, Dublin, OH (US)

Correspondence Address: STANDLEY LAW GROUP LLP, 495 METRO PLACE SOUTH, SUITE 210, DUBLIN, OH, 43017, US

(21) Appl. No.: 11/771,010

(22) Filed: Jun. 29, 2007

Related U.S. Application Data
(60) Provisional application No. 60/817,463, filed on Jun. 29, 2006.

(51) Int. Cl. A61B 17/58; A61B 17/56

(52) U.S. Cl. 606/69; 606/61; 606/73

(57) ABSTRACT

A locking system for use in spinal implantation has a plate member with a screw-receiving hole and a bone screw with a shank threaded at one end and having a head at the other end. The screw has a primary locking feature for engaging a screw-receiving hole in which it is received as well as a secondary locking feature for engaging the screw-receiving hole while providing a signal that the primary locking feature is engaged. The signal may be tactile, audible, visual or a combination thereof.

### CROSS-REFERENCE TO RELATED APPLICATIONS

This application is a non-provisional of U.S. Ser. No. 60/817,463, filed 29 Jun. 2006, which is incorporated by reference as if fully recited herein.

### TECHNICAL FIELD

The disclosed embodiments relate generally to implantable medical devices and the use of such devices in methods for stabilizing skeletal bone. One embodiment relates more particularly to implantable medical devices fabricated of metals and to the use of such devices for stabilizing the vertebrae of a human spine.

### BACKGROUND OF THE ART

A complex arrangement of joints, ligaments, tendons and muscles maintain a normal spinal column in place. Deformities, trauma, degenerative and other diseases may cause abnormal conditions. These conditions generally cause pain in the vertebral joints and/or displacement or rotation of a vertebra relative to the adjacent vertebra. When surgery is needed, the intervertebtal discs and/or vertebrae may be replaced with implants that will hold adjacent vertebrae together. Implants provide fixation or stabilization, which maintains the vertebral position while healing takes place. While originally introduced as crude plates, rods, and screws, spinal implant devices have been developed into sophisticated appliances, including vertebral disc replacements. Rods plates and other spinal implants have taken on various configurations including hybrid designs. These spinal implants attach to the spine, frequently through the use of bone screws.

As with almost any screw, bone screws can inadvertently “back out” from their proper location in the spinal implant. As many spinal procedures utilize an anterior approach to the spine, a “backed out” screw head exposes nearby internal bodies, such as the aorta and the esophagus, to the risk of injuries. This problem has led to the development of devices for locking these bone screws to the spinal implant. Such locking devices not only prevent bone screw back out, but they also reduce the tendency of the screw head to pivot abnormally within the spinal implant. These bone screw locking devices can contain many intricate components that complicate the implantation method, increase the cost of the manufacturing the device, and reduce the reliability of the device. Use of a taper lock device provides a simple, low cost and sturdy device as well as a simple method for locking a bone screw to a spinal implant. These locks are currently in use.

One problem with taper locks used in spinal surgery is that the implanting surgeon may not be able to tell that the taper lock is fully engaged. If the taper lock is not fully engaged, then the bone screw may back out from the spinal implant unexpectedly. Further, the surgery involved in placing the spinal implant is not inconsequential, and the screw placement is not readily monitorable after. Thus, there is a need in the art for a taper lock device that adequately notifies and/or permits a surgeon of proper engagement.

### SUMMARY OF THE INVENTION

Implantable medical devices and the use of such devices for stabilizing skeletal bone are well-known. One embodiment of the present invention relates more particularly to implantable medical devices fabricated of metals and to the use of such devices for stabilizing the vertebrae of a human spine. This embodiment is directed to a method and/or device that involves an improvement for or in the use of a fixing plate and bone screws fabricated from metals. In such an embodiment, one or more bone screws maintain the fixing plate in contact with one or more vertebrae, where the one or more bone screws have a tapered shape at one end thereof. The tapered head of the one or more bone screws is driven into a matching tapered hole of the fixing plate. This results in locking the screw to the plate (taper lock). Once engaged, the taper lock prevents the screw from backing out of the fixing plate and maintains proper orientation of the bone screw head to the fixing plate. The taper(s) used for locking may be within the structure of the fixing plate and bone screw, or within an insert placed into/onto the fixing plate or bone screw. There may be one or more tapers in the fixing plate and on the bone screw. Taper lock devices themselves are not the focus of this application, however, the taper lock must be discussed to describe how the improved method and device works.

The method and/or device of the present invention yields an improved fixing plate/screw taper lock device by: (1) indicating to the implanting surgeon when the taper is fully engaged; and (2) preventing retro-pulsion of the screw, which could disengage the taper lock if the tapers between the bone screw and the fixing plate are not fully engaged.

One object is to provide a method for a spinal fusion, fixation and/or for implantation. Another object is to provide a stabilizer device, which is effective yet uncomplicated mechanically, simple to manufacture, and simple to implant. Still another object of the present invention is to provide a spinal fusion or spinal stabilization device using metallic stabilization plates and plate attachment means. Yet another object of the present invention is to provide devices and methods for cervical, thoracic, and lumbar spinal implants anteriorly, posteriorly, and/or laterally. Still yet another object of the present invention is to provide an improved method and device for locking a bone screw to a spinal fixing plate.

### BRIEF DESCRIPTION OF THE DRAWINGS

A better understanding of the embodiments will be had by reference to the appended drawings and the associated description, where identical parts are identified by identical reference numbers and wherein:

FIG. 1 is a cross-section view of a first embodiment system providing a secondary locking feature; and

FIG. 2 is a cross-sectional view of a second embodiment system providing a secondary locking feature.

### DETAILED DESCRIPTION OF A PREFERRED EMBODIMENT

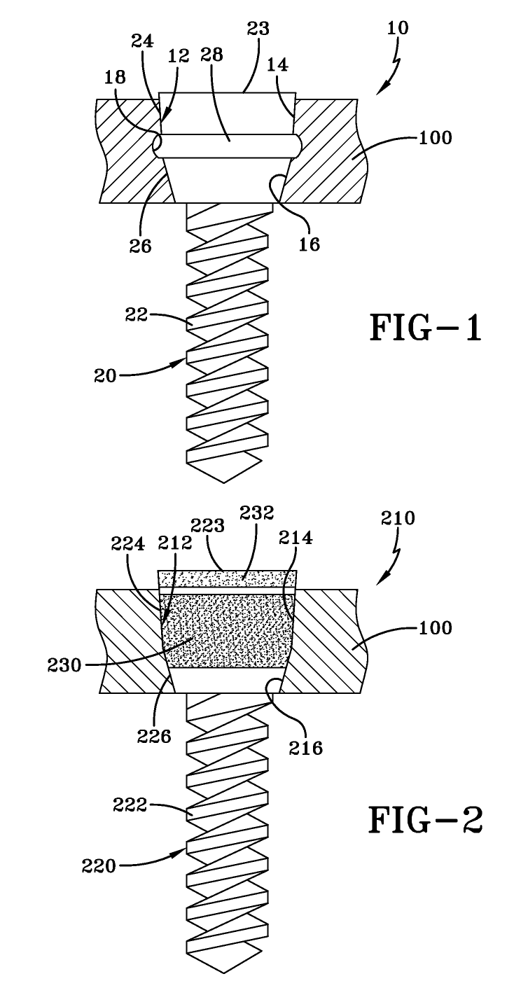

FIG. 1 shows a side sectional view of an implantable medical device 10 for stabilizing skeletal bone. As the devices are well known and the applicability of the improvement disclosed herein is generally not affected by the particular device, the device is illustrated generally as a sectioned body 100, through which a screw-receiving hole 12 is provided. In this embodiment, the screw-receiving hole 12 has a first and a second taper 14, 16, the second taper having a greater angularity relative to a longitudinal axis of the screw-receiving hole than the first taper. In this way, the hole 12 is particularly adapted to receive a screw 20, which has a threaded shank 22 for engaging bone and, at an end of the shank, a head 23. Screw head 23 has two different tapers, the first screw taper 24 corresponding to the receiving hole taper 14 and the second screw taper 26 corresponding to the receiving hole taper 26, in order to provide an interference taper lock, in a manner known in the art. To this point, therefore, the device 10 corresponds to a securing system as would be known in the prior art, the taper lock provided by tapers 24, 26 on the screw 20 effecting a primary locking feature.

The improvement provided by the embodiment lies in the coordination of features seen on both the screw 20 and the screw-receiving hole 12. Particularly, the screw-receiving hole 12 has a circumferential groove 18 formed at a junction of the respective receiving hole holes 14, 16, and the screw 20 has a ring 28 of an expandable material fixedly positioned on the head 23, at a junction of the different screw head tapers 24, 26. The ring 28 may be metallic, polymeric or some combination of metallic and polymeric. The ring 28 should elastically deform and restore its shape upon engaging a structural feature of the screw-receiving hole 12, especially the groove 18 and the transition from taper 14 to the groove 18.

The elastic deformation and restoration of the ring 28 may cause an audible indication, especially a “click,” that provides a positive indication of engagement of the ring, which serves as a secondary locking feature. Because of the position of the groove 18 and the ring 28 on the screw head 23, this audible indication also signals the engagement of the primary locking feature.

In addition to, or in lieu of, the audible signal, the elastic deformation and restoration of the ring 28 may cause a tactile indication to the surgeon implanting the device, as the restoration may effect a vibration in the driving tool being used to place the screw. Again, the position of the groove 18 and the ring 28 on the screw head 23 are such that the tactile indication also signals the engagement of the primary locking feature.

In some of the implementations of this embodiment, the flexible material of ring 28 will be colored, particularly with a color that will be readily distinguishable in the operative field of use. For example, a cobalt blue color or a green color would not be normally encountered in bodily tissues and structures involved in a spinal implant procedure, so a flexible ring having such a color would be particularly notable to a surgeon. As the flexible ring 28 is seated in the groove 18, the color of the ring would be obscured from view, providing a visible indication of engagement.

In this embodiment 10, the secondary locking feature provided by ring 28 and groove 18 assists the engagement by the primary locking feature of the screw in the screw-receiving hole, although the secondary locking feature is generally designed to indicate positive engagement more than to provide strong engagement. For this reason, the secondary locking feature would generally not be used in the absence of a primary locking feature.

FIG. 2 shows a side sectional view of an implantable medical device 210 for stabilizing skeletal bone. As the devices are well known and the applicability of the improvement disclosed herein is generally not affected by the particular device, the device is illustrated generally as a sectioned body 100, through which a screw-receiving hole 212 is provided. In this embodiment, the screw-receiving hole 212 has a first and a second taper 214, 216, the second taper having a greater angularity relative to a longitudinal axis of the screw-receiving hole than the first taper. In this way, the hole 212 is particularly adapted to receive a screw 220, which has a threaded shank 222 for engaging bone and, at an end of the shank, a head 223. Screw head 223 has two different tapers, the first screw taper 224 corresponding to the receiving hole taper 214 and the second screw taper 226 corresponding to the receiving hole taper 226, in order to provide an interference taper lock, in a manner known in the art. To this point, therefore, the device 210 corresponds to a securing system as would be known in the prior art, the taper lock provided by tapers 224, 226 on the screw 220 effecting a primary locking feature.

The improvement provided by this embodiment lies in the coloration of screw head 223. Particularly, a band of first coloration 230 is placed on the screw head 223, with a band of a second coloration 232 circumferentially placed closer to the driving face of the screw 220. In some embodiments, the second band 232 may be simply a retention natural color of the screw. In either case, the first coloration is selected to be a color that will be readily distinguishable in the operative field of use, in the same manner described with regard to the first embodiment. The first band 230 of coloration will be carefully positioned on the screw head 23 so that positive engagement of the primary locking feature is indicated when the entire first band has been obscured by the screw-receiving hole 212.

Variations and modifications of the present invention will be obvious to those skilled in the art and the present invention is intended to cover in the appended claims all such modifications and equivalents.

What is claimed is:

1. A improved locking system for use in spinal implantation, comprising a plate member with a screw-receiving hole; a bone screw with a shank threaded at one end and having a head at the other end, the screw provided with a primary locking feature for engaging a screw-receiving hole in which it is received, the improvement comprising:
a secondary locking feature for engaging the screw-receiving hole while providing a signal that the primary locking feature is engaged, the signal being at least one of: a tactile, audible and visual signal.

2. The improved locking system of claim 1, wherein:
the secondary locking feature is a ring of flexible material formed in a circumferential groove in the screw head.

3. The improved locking system of claim 2, wherein:
the flexible material has a coloration that is readily distinguishable in the operative field of use and engagement of the screw in the screw-receiving hole obscures the flexible material, providing a visible indication of engagement.

4. The improved locking system of claim 2, wherein:
the ring elastically deforms and restores upon engaging a structural feature of the screw-receiving hole, causing an audible indication of engagement.

5. The improved locking system of claim 2, wherein:
the flexible material elastically deforms and restores upon engaging a structural feature of the screw-receiving hole, causing a tactile indication of engagement, transmitted through the screw to a device driving the screw.

6. The improved locking system of claim method of claim 1, wherein the secondary locking feature assists the engagement by the primary locking feature of the screw in the screw-receiving hole.

7. An improved method affixing a spinal implant to a vertebral body, comprising the steps of:
providing an improved locking system comprising a plate member with a screw-receiving hole; a bone screw with a shank threaded at one end and having a head at the other end, the screw provided with a primary locking feature for engaging a screw-receiving hole in which it is received and a secondary locking feature for engaging the screw-receiving hole while providing a signal that the primary locking feature is engaged, the signal being at least one of: a tactile, audible and visual signal;
positioning the plate member on the vertebral body; and
driving the bone screw into the vertebral body through the screw-receiving hole to depth sufficient to engage the primary locking feature and to engage the secondary locking feature, the latter engagement being confirmed by the signal.
